(19) United States
(12) Patent Application Publication
(10) Pub. No.: US 2020/0211711 A1
(43) Pub. Date: Jul. 2, 2020

(54) HEALTH CONDITION DETERMINATION SYSTEM, HEALTH CONDITION DETERMINATION METHOD, AND RECORDING MEDIUM

(71) Applicant: Konica Minolta, Inc., Tokyo (JP)

(72) Inventor: Tsuyoshi KOBAYASHI, Tokyo (JP)

(21) Appl. No.: 16/707,565

(22) Filed: Dec. 9, 2019

(30) Foreign Application Priority Data
Dec. 28, 2018 (JP) 2018-246645
Oct. 8, 2019 (JP) 2019-185162

(51) Int. Cl. G16H 50/30; G16H 50/20; A61B 5/00; A61B 5/0205

(52) U.S. Cl. CPC G16H 50/30; G16H 50/20; A61B 5/7275; A61B 5/4815; A61B 5/0004; A61B 5/4812; A61B 5/0205

(57) ABSTRACT

A health condition determination system includes a measurement device that measures a vital value of a subject person, a first hardware processor that generates model information indicating daily circadian rhythm of the subject person from the vital value measured by the measurement device for one day or more, and a second hardware processor that compares a vital value measured by the measurement device after generation of the model information with the model information to determine the presence of a disturbance occurrence in the circadian rhythm of the subject person based on the comparison result.

### CROSS-REFERENCE TO RELATED APPLICATIONS

The entire disclosure of Japanese Patent Applications No. 2018-246645 filed on Dec. 28, 2018 and No. 2019-185162 filed on Oct. 8, 2019 is incorporated herein by reference in its entirety.

### BACKGROUND

### Technological Field

The present invention relates to a health condition determination system, a health condition determination method, and a recording medium.

### Description of the Related Art

In recent years, a technique has been developed in which a measurement device acquires biological information useful for human health management, such as pulse rate and body temperature, and the acquired biological information is used to determine the health condition.

For example, JP 6338298B discloses a device that obtains data for determination by substituting measured values of body surface temperature, pulse rate, respiration, and blood oxygen concentration into a predetermined determination formula and calculates body conditions of a subject person.

### SUMMARY

However, the device disclosed in JP 6338298B determines health (normal) or non-health (abnormal) in light of general and common indices, and therefore, cannot evaluate individual differences. Therefore, the accuracy in determination is lower. For example, when a subject person constantly has a higher body temperature, there is a tendency that abnormality values are constantly output. Further, since this conventional device requires four types of vital values (body surface temperature, pulse rate, respiration, and blood oxygen concentration) in the determination, securing the measurement device is difficult, installation thereof is complicated, and processing is complicated.

An object of the present invention is to provide a health condition determination system, a health condition determination method, and a recording medium, which can determine the health condition of a subject person more accurately and simply.

To achieve at least one of the abovementioned objects, according to an aspect of the present invention, health condition determination system reflecting one aspect of the present invention comprises:

a measurement device that measures a vital value of a subject person; and

a hardware processor that generates model information indicating daily circadian rhythm of the subject person from the vital value measured by the measurement device for one day or more, compares a vital value measured by the measurement device after generation of the model information with the model information, and determines the presence of a disturbance occurrence in the circadian rhythm of the subject person based on the comparison result.

To achieve at least one of the abovementioned objects, according to another aspect of the present invention, a health condition determination method reflecting one aspect of the present invention comprises:

generating model information indicating daily circadian rhythm of a subject person from a vital value measured for one day or more by a measurement device that measures the vital value of a subject person; and

comparing a vital value measured by the measurement device after generation of the model information with the model information and determining the presence of a disturbance occurrence in the circadian rhythm of the subject person based on the comparison result.

To achieve at least one of the abovementioned objects, according to another aspect of the present invention, a recording medium reflecting one aspect of the present invention is a non-transitory recording medium that stores a computer-readable program that causes a computer to function as:

a hardware processor that generates model information indicating daily circadian rhythm of a subject person from a vital value measured for one day or more by a measurement device that measures the vital value of the subject person, compares a vital value measured by the measurement device after generation of the model information with the model information, and determines the presence of a disturbance occurrence in the circadian rhythm of the subject person based on the comparison result.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are no intended as a definition of the limits of the present invention, wherein:

FIG. 1 is a diagram illustrating a schematic configuration of a health condition determination system;

FIG. 2 is a diagram illustrating a functional configuration of a management device;

FIG. 3 is a diagram summarizing a general state in a compensated period and a decompensated period at the time of health and at the time of disease;

FIG. 4 is a flowchart illustrating an exemplary flow of first circadian rhythm comparison processing;

FIG. 5A is a diagram illustrating exemplary model information;

FIG. 5B is a diagram illustrating exemplary model information;

FIG. 6 is a flowchart illustrating an exemplary flow of second circadian rhythm comparison processing;

FIG. 7 is a flowchart illustrating an exemplary flow of third circadian rhythm comparison processing;

FIG. 8 is a diagram illustrating exemplary vital values in a depressed state;

FIG. 9 is a flowchart illustrating an exemplary flow of correlation information comparison processing;

FIG. 10 is a diagram illustrating exemplary second model information;

FIG. 11 is a diagram illustrating an exemplary change in vital values when the physical condition deteriorates;

FIG. 12 is a conceptual diagram illustrating how the correlation information changes;

FIG. 13 is a flowchart illustrating an exemplary flow of specific symptom detection processing; and

FIG. 14 is a diagram illustrating exemplary state in a terminal period.

### DETAILED DESCRIPTION OF THE EMBODIMENTS

Hereinafter, embodiments of the present invention will be described in detail below with reference to attached drawings. However, the scope of the invention is not limited to the disclosed embodiments.

### [Configuration of Health Condition Determination System]

First, an exemplary configuration of a health condition determination system 100 according to the present embodiment will be described.

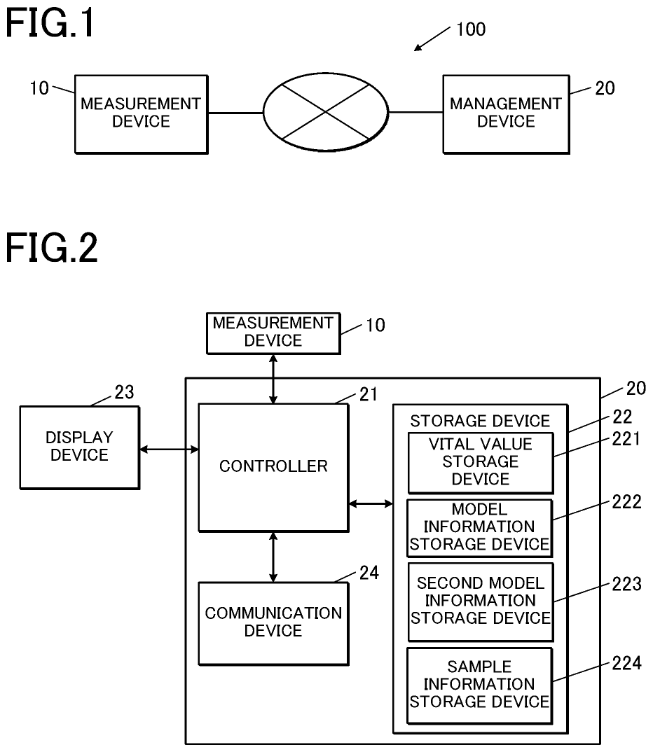

FIG. 1 is a diagram illustrating a schematic configuration of the health condition determination system 100.

As illustrated in FIG. 1, the health condition determination system 100 is configured to include, for example, a measurement device 10 (measurement device) and a management device 20. These devices are configured to be able to communicate with each other via a wired or wireless communication network.

The measurement device 10 is, for example, a device configured to measure vital values of a subject person regardless of contact/non-contact with a subject body. Specifically, the measurement device 10 is, for example, a device having measurement functions of a measurement instrument, such as a thermometer, a blood pressure monitor, a respiratory meter, a heart rate monitor, a pulse oximeter, a gravitational accelerometer, a blood glucose meter, or a camera image analyzer. The measurement device 10 is suitably selected with respect to type, shape, and measurement function according to a subject person. The measurement device 10 may have a function of displaying measured vital values.

The measurement device 10 measures vital values or values comparable to them of a subject person using the measurement function and transmits the measured values to the management device 20.

The vital values are, for example, biological information including body temperature, blood pressure (contraction, diffusion), respiration (respiration rate, respiration rhythm), heart rate, pulse rate, heart rate pulse rhythm, SpO2 (oxygen saturation), blood sugar level, acceleration (a value representing the body movement). The values comparable to the vital values are, for example, biological information such as body surface temperature, blood pressure value by a cuff-less blood pressure monitor, voltage/radio wave type noncontact vital sensor value, autonomic nerve index value.

The management device 20 is a device that accumulates vital values transmitted from the measurement device 10 and performs processing for determining the health condition of a subject person using the vital values (as described in detail below).

The subject person whose health condition can be determined by the health condition determination system 100 is not particularly limited by age or gender. For example, the subject person may be a person who is temporarily in a poor physical condition due to illness or the like and is expected to recover, or a person who is in or near a terminal period such as an elderly person.

Further, the subject person is not limited to a person being in a medical institution such as a hospital or a clinic, and may be in a subject person's home located away from a medical institution.

The configurations of the measurement device 10 and the management device 20 can be appropriately set according to the state of the subject person and the locations of the subject person and the medical institution.

For example, when the subject person is hospitalized in a medical institution, the measurement device 10 and the management device 20 may be integrated. When the subject person is bedridden at home, the measurement device 10 and the management device 20 may be separate and configured to be communicably connected via a wireless communication network.

FIG. 2 is a diagram illustrating a functional configuration of the management device 20.

As illustrated in FIG. 2, the management device 20 is configured to include, for example, a controller 21 (hardware processor), a storage device 22, a display device 23, a communication device 24, which are connected via a bus.

The controller 21 is configured by a central processing unit (CPU), a Read Only Memory (ROM), a random access memory (RAM) and the like, and totally controls processing operations of respective components of the management device 20. The CPU reads various processing programs stored in the ROM and develops them in the RAM to execute various processing according to the developed program.

The storage device 22 is constituted by a storage device, such as a nonvolatile semiconductor memory or a hard disk, and stores data related to various processing.

Specifically, the storage device 22 includes, for example, a vital value storage device 221, a model information storage device 222, a second model information storage device 223, and a sample information storage device 224.

The vital value storage device 221 stores the vital values of the subject person measured and transmitted by the measurement device 10 in time series for each type.

The model information storage device 222 stores model information indicating a referential daily circadian rhythm of the subject person, which has been generated by acquiring a vital value of the subject person for a predetermined learning period of one day or more.

The second model information storage device 223 stores, as second model information, correlation information indicating the correlation between at least two types of referential vital values of the subject person, which have been generated by acquiring at least two types of vital values of the subject person.

The sample information storage device 224 stores in advance, as sample information, various vital values of numerous persons in association with characteristics (gender, age, medical condition, etc.) of respective persons. The sample information may include vital values of the subject person.

The sample information also includes vital values indicating specific symptoms that appear in the terminal period. Further, the sample information includes disease information indicating a pattern of changes in the vital values appearing in the terminal period for each disease. The disease information may include disease identifiable information acquired from an electronic medical record or the like in addition to the pattern of changes of the vital value.

The display device 23 is provided as a separate device and includes, for example, a liquid crystal display (LCD) on which various screens or the like can be displayed according to instructions of the controller 21.

The communication device 24 performs transmission/reception of data to/from an external device, such as the measurement device 10, connected to the communication network.

### [Operations of Health Condition Determination System]

Next, operations of the health condition determination system 100 will be described.

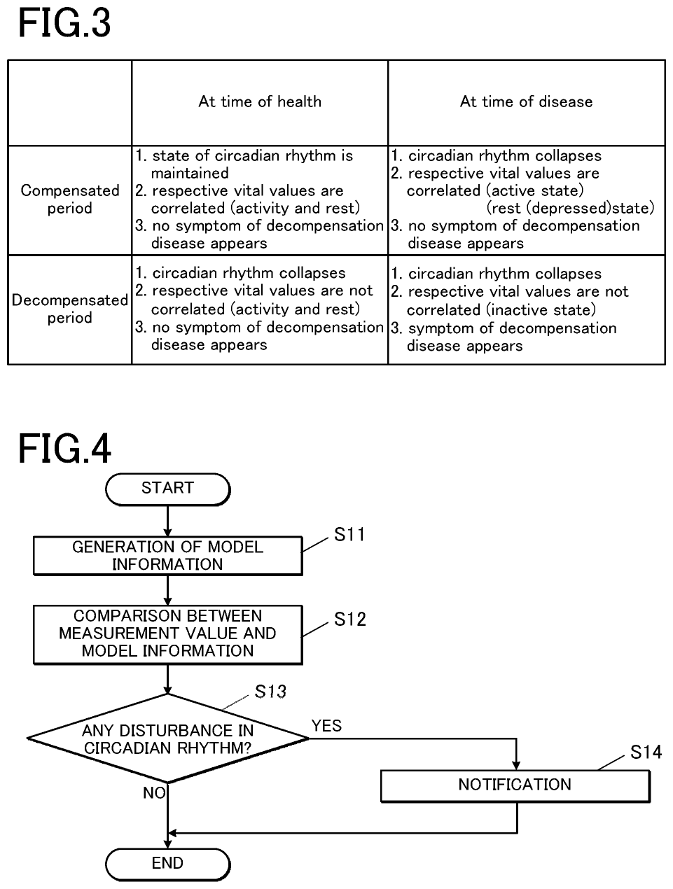

FIG. 3 is a diagram summarizing a general human body state in a non-terminal period (“compensated period” in medical term) and in a terminal period (“decompensated period” in medical term) at the time of health and at the time of disease. As illustrated in FIG. 3, it is possible to grasp the state of the subject person from the circadian rhythm or the tendency of vital values.

In the health condition determination system 100, the measurement device 10 measures vital values of a subject person constantly or periodically and transmits the measured vital values to the management device 20.

The management device 20 receives the vital values transmitted from the measurement device 10, and executes health condition determination processing for determining the health condition of a subject person based on the vital values.

The health condition determination processing includes circadian rhythm comparison processing, correlation information comparison processing, and specific symptom detection processing.

The circadian rhythm comparison processing is processing for determining whether the subject person is healthy from the disturbance in the circadian rhythm of the subject person.

The correlation information comparison processing is processing for calculating the degree of decline in vital functions of the subject person from the disturbance in the correlation between the vital values of the subject person.

The specific symptom detection processing is processing for estimating the date of death by detecting the occurrence of a specific symptom from the vital values of the subject person.

Combining these tree types of processing can determine whether the subject person is healthy, or whether there is a deterioration in the vital functions of the subject person, or whether the end of life of the subject person being in the terminal period is approaching closely.

Hereinafter, each of the above-mentioned processing will be described in detail.

### <Circadian Rhythm Comparison Processing>

The circadian rhythm is also referred to as a biological clock and is a physical rhythm that fluctuates in a cycle of about 24 hours. The circadian rhythm repeats a constant regular rhythm at the time of health, but it collapses at the time of disease.

In the circadian rhythm comparison processing, the health condition of a subject person is determined from the degree of deviation at the time of measurement with respect to the referential circadian rhythm (model information) of the subject person.

### (First Circadian Rhythm Comparison Processing)

FIG. 4 is a flowchart illustrating an exemplary flow of the first circadian rhythm comparison processing.

The first circadian rhythm comparison processing is processing in which a subject person is assumed to be living a regular life.

As illustrated in FIG. 4, first, the controller 21 generates model information indicating daily circadian rhythm of the subject person (model information generation process: step S11).

Specifically, the controller 21 acquires vital values (fluctuation vertical range) from the time of awakening to the time of sleeping of the subject person measured by the measurement device 10 for a learning period of one day or more, and generates the daily circadian rhythm of the subject person as the model information. For example, when a heart rate monitor is used as the measurement device 10 to acquire daily vital values representing the heart rate, heart rate rhythm, and autonomic nerve, the controller 21 can generate a plurality of pieces of model information representing circadian rhythms of these values (heart rate, heart rate rhythm, and autonomic nerve).

If the learning period spans multiple days, the model information can be generated by calculating an average value thereof and variations so that the accuracy can be enhanced. On the other hand, if it is desired to start the subsequent determination processing as soon as possible, a learning period of at least one day will be enough.

In addition, when generating the model information, it is preferable to have state information at the time of awakening (be active) and the time of sleeping (rest) because the accuracy can be enhanced. However, if these values cannot be measured, time zone (daytime and nighttime) may be taken into consideration in acquiring the vital values.

Further, although the model information to be generated is not limited in total number (that is, the number of vital values to be used is not limited), using a plurality of data will be able to enhance the accuracy.

The generated model information is stored in the model information storage device 222.

Further, when generating the model information, it is desired that the subject person is healthy.

As described above, the model information can be generated in a learning period of at least one day. However, if it is not known whether the subject person is healthy in one day corresponding to the learning period, the accuracy may be deteriorated in the subsequent determination processing.

Therefore, it may be possible to acquire vital values of the subject person over five days to ten days, obtain a correlation value of the circadian rhythm every two days, and determine whether the subject person is healthy from the fluctuation of the correlation value. In other words, the correlation value of the circadian rhythm is obtained for each period of the first day and the second day, the second day and the third day, the third day and the fourth day . . . . Then, when the state in which the obtained correlation value is within a permissible range (for example, within ±0.2) continues for a predetermined number of days or more, it is determined that the subject person is healthy. In this case, the model information can be generated from the average value in a period in which the subject person is determined as being healthy.

It is preferable to store the correlation value of each individual subject person being in a healthy condition obtained in this case as a health index value indicating the health condition. For example, since the health index value changes due to the collapse of the relationship between vital values at the end of life, it is possible to grasp this timing as a quantitative value.

Next, in response to reception of the vital values (measurement values) measured by the measurement device 10 and transmitted at predetermined timing, the controller 21 compares the measurement values with the model information (determination process: step S12).

Specifically, the controller 21 compares the measurement values measured by the measurement device 10 and transmitted at every predetermined timing with data of the model information at a time corresponding to the predetermined timing, and then calculates difference values.

Further, the controller 21 may compare measurement values measured and transmitted at the time of awakening (be active) and the time of sleeping (rest) with data of the model information corresponding to the time of awakening (be active) and the time of sleeping (rest), and then calculate difference values.

Further, when the measurement values for one day are accumulated, the controller 21 may compare the measurement values for one day with the model information and calculate a deviation amount from the model information per day.

Further, if there is a plurality of types of measurement values, the measurement values are compared with corresponding model information.

Next, the controller 21 determines whether a disturbance has occurred in the circadian rhythm of the subject person based on the comparison result (determination process: step S13).

Specifically, the controller 21 determines that the disturbance has occurred in the circadian rhythm of the subject person when the difference value of the measurement value at predetermined timing from the model information exceeds a predetermined threshold. When there is a plurality of types of measurement values, the threshold may be set according to the type.

Further, when the difference value from the model information has exceeded the predetermined threshold with respect to at least one of the measurement values at the time of awakening (be active) and the measurement values at the time of sleeping (rest), the controller 21 determines that there is a disturbance having occurred in the circadian rhythm of the subject person.

Further, when the measurement values for one day are accumulated, the controller 21 compares the measurement values for one day with the model information and calculates a deviation amount from the model information per day. If the calculated deviation amount has exceeded the predetermined threshold, it is determined that the disturbance has occurred in the circadian rhythm of the subject person.

Specifically, for example, when the measurement values for one day are compared with the model information and the period in which the amplitude is halved (i.e., the deviation amount) exceeds the threshold (e.g., 30%), it is determined that there is a disturbance having occurred in the circadian rhythm (abnormal).

When the measurement values are accumulated for two days or more, the difference value and the deviation amount can be calculated from the average and variation of the model information per day. In other words, a circadian rhythm variation (σ) can be calculated beforehand as model information by mean square error square root (RMSE), and it can be determined that there is a disturbance having occurred in the circadian rhythm (abnormal) when the circadian rhythm variation at the time of measurement has exceeded σ. This determination can be applied to determination at predetermined timing, determination using data at the time of awakening (be active) and the time of sleeping (rest), and determination when the measurement values for one day are accumulated.

Further, it may be determined that the disturbance has occurred in the circadian rhythm of the subject person from the difference value of a plurality of measurement values or the total value of the deviation amount. In this case, weighting depending on the type of the vital value may be desired.

Hereinafter, a specific example of the comparison result is described below.

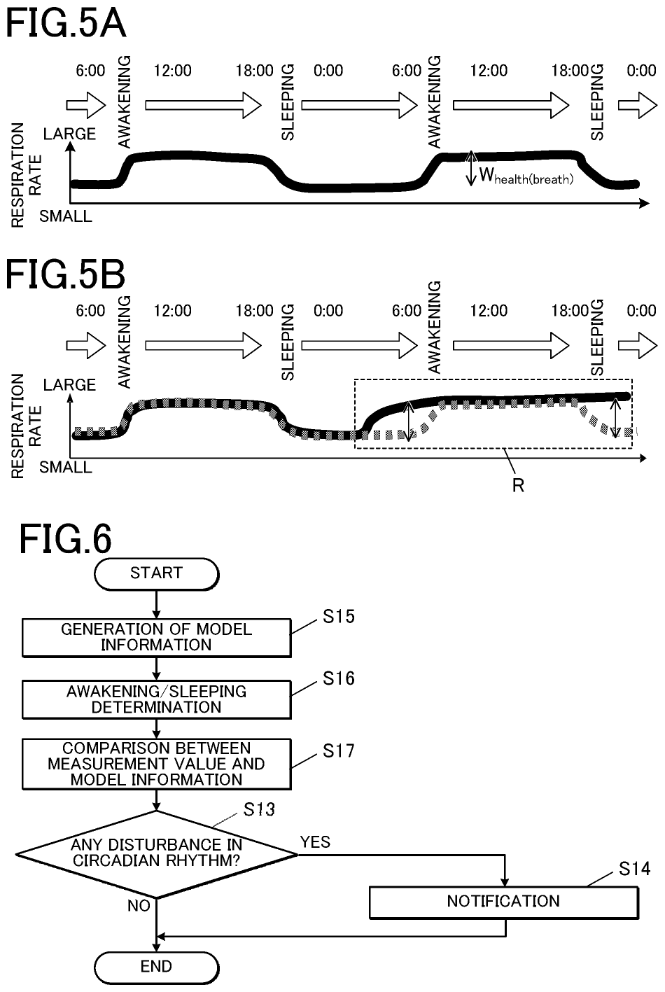

FIG. 5A illustrates exemplary model information. FIG. 5B illustrates an exemplary comparison result between the model information (dashed line) and the measurement value (solid line). Here, the vital value is “respiration rate”. The horizontal axis represents time and the vertical axis represents the magnitude of the respiration rate.

As illustrated in FIG. 5A, it can be confirmed that the circadian rhythm of the model information cyclically varies at a constant period. In other words, the respiration rate fluctuates periodically according to rising and sleeping.

On the other hand, in FIG. 5B, it can be confirmed that there is a deviation from the model information in a region R.

Further, if it is determined that no disturbance in the circadian rhythm has occurred (normal) (NO in step S13), the controller 21 terminates the present processing.

On the other hand, if it is determined that there is a disturbance having occurred in the circadian rhythm (abnormal) (YES in step S13), the controller 21 causes the display device 23 to display a message or notifies this by flickering light or issuing an alarm (step S14), and terminates the present processing.

According to the above-described circadian rhythm comparison processing, since the data of each individual subject person is used in the comparison, it can be accurately determined whether the health condition of the subject person is normal or abnormal.

Further, since the determination is feasible with only one vital value, processing can be performed more easily. In addition, since the duration required in the generation of model information for comparison is at least one day, processing can be performed more easily.

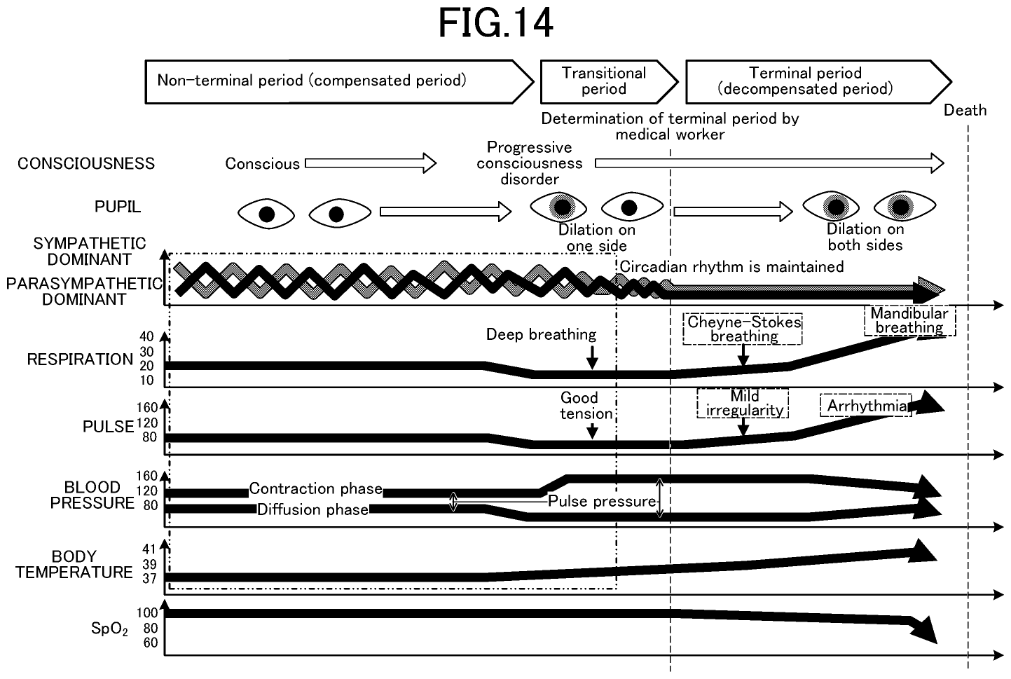

Since the circadian rhythm is disturbed due to deterioration of vital functions (see an area surrounded by a two-dot chain line in FIG. 14), the circadian rhythm measured in the circadian rhythm comparison processing can also be used for determining whether it is the terminal period.

### (Second Circadian Rhythm Comparison Processing)

FIG. 6 is a flowchart illustrating an exemplary flow of the second circadian rhythm comparison processing.

The second circadian rhythm comparison processing is processing in which the subject person is assumed to be an irregular habitant living an irregular life.

The second circadian rhythm comparison processing makes it possible to accurately determine the health condition even for the irregular habitant by using sleep determination (whether it is the time of awakening (be active) or the time of sleeping (rest)).

As illustrated in FIG. 6, the controller 21 generates model information indicating daily circadian rhythm of the subject person by the same method as in step S11 of the first circadian rhythm comparison processing (step S15).

In step S15, model information of the subject person corresponding to each of the time of awakening and the time of sleeping (REM/NONREM) is generated.

Next, when the controller 21 receives vital values (measurement values) measured and transmitted by the measurement device 10 at predetermined timing, the controller 21 determines the state of the subject person (the time of awakening or the time of sleeping) based on the measurement values (step S16).

Specifically, the controller 21 measures a stress index (LF/HF) from the heart rate interval of the vital values, and determines whether it is the time of awakening or the time of sleeping (REM/NONREM). In the determination, body movement information can also be used.

Next, the controller 21 selects model information that matches the state of the subject person and compares the received measurement values with the selected model information (step S17). Subsequently, the processing proceeds to step S13 similar to that in the first circadian rhythm comparison processing.

In the first circadian rhythm comparison processing, it is determined whether the health condition of the subject person is normal or abnormal based on the circadian rhythm. However, if the subject person is an irregular habitant living an irregular life, such as days and nights being reversed due to life style or the like or sleepless night due to fatigue or the like, the circadian rhythm may collapse and performing accurate determination may be difficult.

On the other hand, according to the second circadian rhythm comparison processing, the model information that matches the life of the subject person is selected and compared with the measurement values. Therefore, even when the subject person is an irregular habitant, it can be accurately determined whether the health condition is normal or abnormal.

### (Third Circadian Rhythm Comparison Processing)

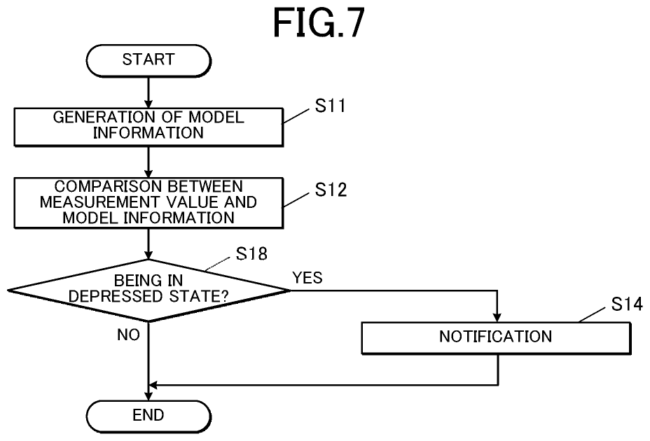

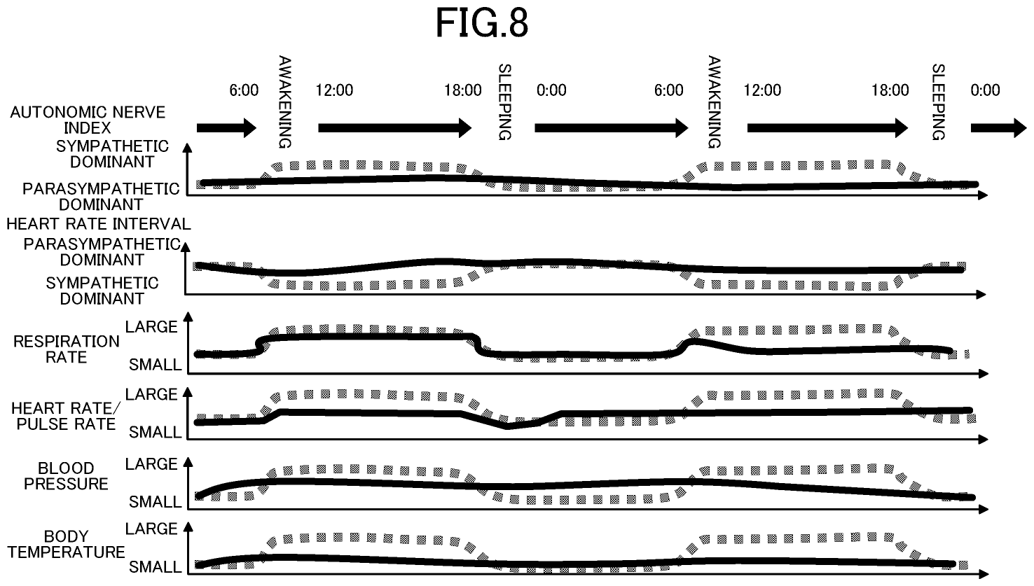

FIG. 7 is a flowchart illustrating an exemplary flow of the third circadian rhythm comparison processing.

The third circadian rhythm comparison processing is processing that can be used when the subject person is suspected of being depressed.

As illustrated in FIG. 7, in the same manner as in the first circadian rhythm comparison processing, the controller 21 generates model information (step S11) and compares the measurement values with the model information (step S12).

Next, the controller 21 determines whether the subject person is in a depressed state (step S18). If the determination result is affirmative (YES in step S18), the controller 21 notifies this (step S14) and terminates the present processing.

FIG. 8 illustrates exemplary data indicating the stress index (LF/HF) in active periods and rest periods of a patient being in a depressed state, measured from the heart rate interval of the vital values.

In general, the body enters an active state when suffering from a disease, but it becomes resting when suffering from depression. In the case of not suffering from depression, the active periods and the rest periods are clearly discriminable. However, as illustrated in FIG. 8, in the case of suffering from depression, it can be understood that parasympathetic nerves get excited even though they should be in the active period (at the time of awakening).

In step S18, it can be determined that the patient is in a depressed state from the tendency of these measurement values.

According to the above-described third circadian rhythm comparison processing, it is possible to determine the depressed state of the subject person. Especially, determining the state of depression is difficult when the subject person is a bedridden patient who has difficulty in expression of intention, and therefore this processing is useful for such patients.

### <Correlation Information Comparison Processing>

The human body has a plurality of types of vital values that are correlated with each other.

The correlation of these vital values is maintained regardless of the healthy or diseased state of the subject person. However, it is known that the correlation collapses if the vital functions of the subject person deteriorate due to serious illness or senility. When the recovery cannot be expected or is not required, it is generally called the terminal period.

In the correlation information comparison processing, a change in the correlation between at least two vital values is used as an index value for the degree of decline in vital functions to enable a medical worker to easily determine whether the subject person is in the terminal period or in the non-terminal period prior to the terminal period.

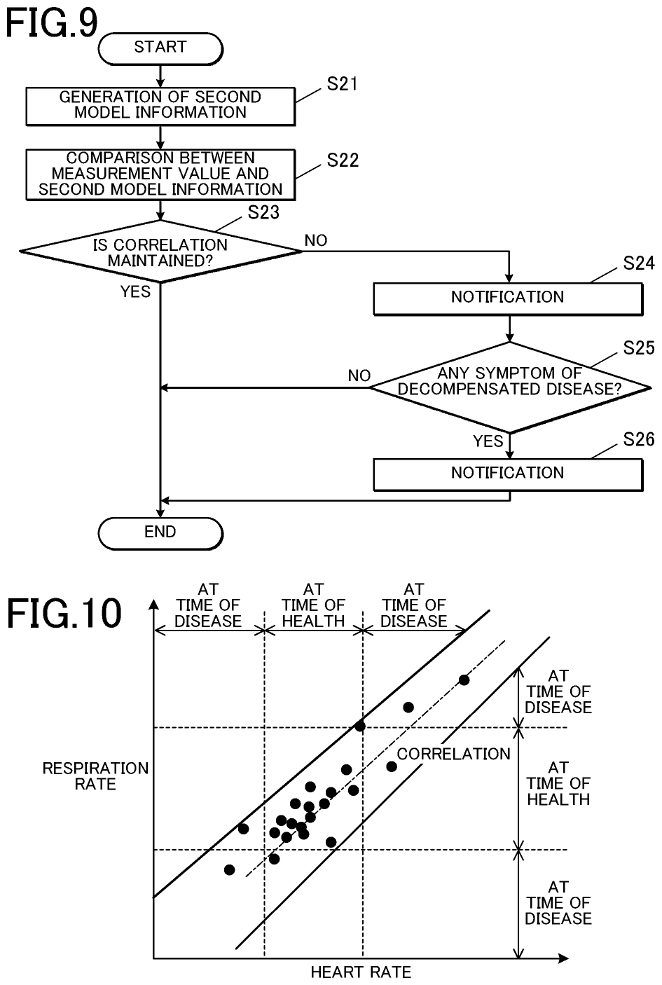

FIG. 9 is a flowchart illustrating an exemplary flow of the correlation information comparison processing.

As illustrated in FIG. 9, first, the controller 21 generates, as second model information, correlation information indicating the correlation between at least two referential vital values of the subject person (step S21).

When generating the second model information, it is desired that the subject person is in the non-terminal period.

Specifically, when the controller 21 acquires at least two vital values measured by the measurement device 10, the controller 21 calculates correlation information such as a correlation value or a regression line. The calculated correlation information is stored in the second model information storage device 223, as the second model information.

For example, the correlation value (r) can be obtained by the following formula (1).

$$\begin{matrix}[ Expression 1 ] & \\ r = \frac{\frac{1}{N} {Σ}_{i = 1}^{N} ( {X}_{i} - \overline{X} ) ( {Y}_{i} - \overline{Y} )}{\sqrt{\frac{1}{N} {{Σ}_{i = 1}^{1} ( {X}_{i} - \overline{X} )}^{2}} \sqrt{\frac{1}{N} {{Σ}_{i = 1}^{1} ( Y - \overline{Y} )}^{2}}} & ( 1 )\end{matrix}$$

Further, the presence of the correlation can be determined in the following manner.

r=0 . . . not correlated

0<|r|≤0.2 . . . almost not correlated

0.2<|r|≤0.4 . . . weakly correlated

0.4<|r|≤0.7 . . . correlated

0.7<|r|<1.0 . . . strongly correlated

r=1.0 or −1.0 . . . perfectly correlated

A reduction in this correlation value indicates that there is a deterioration in vital functions. Therefore, this correlation value is used as an index value indicating the degree of decline in vital functions.

Next, when the controller 21 receives the two vital values measured and transmitted by the measurement device 10, the controller 21 generates correlation information (measurement value) between these two vital values, calculates a comparison result in comparison with the second model information, and displays the calculated comparison result (step S22).

Next, the controller 21 determines whether the difference (the comparison result) between the correlation information (measurement value) of the two vital values and the second model information is equal to or less than a predetermined threshold, that is, whether the correlation is maintained at a degree comparable to the previous one (step S23).

Further, if the difference (the comparison result) is equal to or less than the predetermined threshold, that is, when the correlation is maintained at the degree comparable to the previous one (YES in step S23), the controller 21 terminates the present processing.

On the other hand, if it is determined that the difference (the comparison result) between correlation information (measurement value) of the two vital values and the second model information is greater than the predetermined threshold, that is, when the correlation is weakened compared to the previous one and it is determined that there is a deterioration in vital functions (NO in step S23), the controller 21 causes the display device 23 to display a message or notifies this by flickering light or issuing an alarm (step S24).

As described above, regarding the comparison result, the fact that the correlation is weakened compared to the previous one (the correlation value is reduced) indicates that there is a deterioration in vital functions of the subject person. In other words, it can be determined whether the subject person is in compensated period (i.e., the non-terminal period) or in the decompensated period (i.e., the terminal period).

Hereinafter, a specific example of the comparison result is described below.

FIG. 10 illustrates exemplary correlation information between two vital values (heart rate and respiration).

As illustrated in FIG. 10, in the non-terminal period (compensated period), there is a correlation between two vital values at the time of health. Further, in the non-terminal period (compensated period), the correlation between the two vital values remains unchanged even at the time of disease although the vital values may not the same as those at the time of health due to the circadian rhythm disturbance.

On the other hand, in the terminal period (decompensated period), the correlation between the two vital values collapses (a portion outside a correlation area in FIG. 10).

Medical workers can determine whether it is the terminal period referring to such a change in the correlation between the two vital values.

Referring back to FIG. 9, next, the controller 21 determines whether there is a symptom of a decompensated disease based on the change in correlation information, that is, whether a subject person's physical condition has deteriorated (step S25).

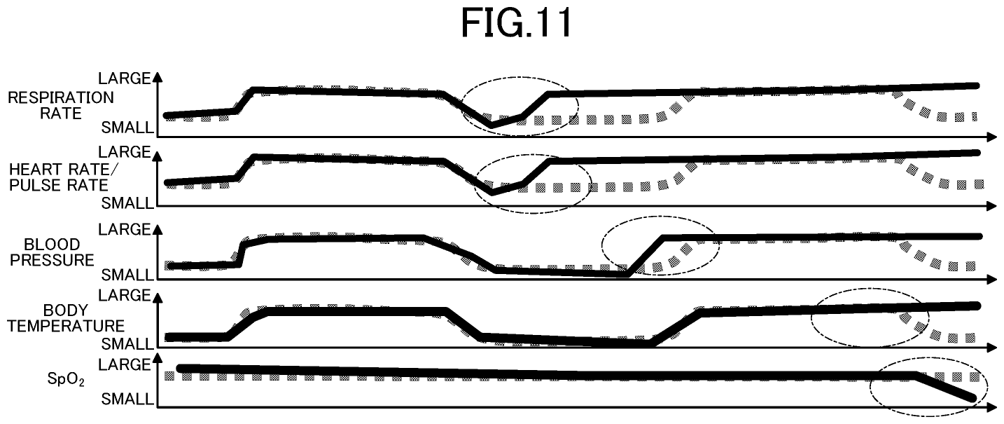

When a certain vital value has changed, correlation information using this vital value changes correspondingly and accordingly it can be indicated that the subject person's physical condition has changed. Examples of the vital values that are likely to change when the physical condition deteriorates are, for example, respiration, heart rate/pulse rate, blood pressure, body temperature, SpO2, as illustrated in FIG. 11. Further, when the correlation value between respiration and heart rate/pulse rate, the correlation value between respiration and body temperature, and the correlation value between body temperature and SpO2 are compared with each other, the change between respiration and heart rate/pulse rate appears most rapidly, the change between respiration and body temperature appears next, and the change between body temperature and SpO2 appears last, in general.

It is possible to determine the deterioration in physical condition at an initial stage based on the tendency of the change of such a plurality of pieces of correlation information.

If there is no symptom of a decompensated disease (NO in step S25), the controller 21 terminates the present processing. On the other hand, if there is a symptom of a decompensated disease, that is, when the subject person's physical condition has deteriorated (YES in step S25), the controller 21 causes the display device 23 to display a message or notifies this by flickering light or issuing an alarm (step S26), terminates the present processing.

In addition, it is possible to calculate an average value according to the following formula (2) from respective correlation values of two vital values in a plurality of different combinations.

$$\begin{matrix}[ Expression 2 ] & \\ {r}_{A v e r a g e ( t )} = \frac{1}{n} ( \vert {r}_{A B ( t )} \vert + \vert {r}_{A C ( t )} \vert + \vert {r}_{B C ( t )} \vert + … ) & ( 2 )\end{matrix}$$

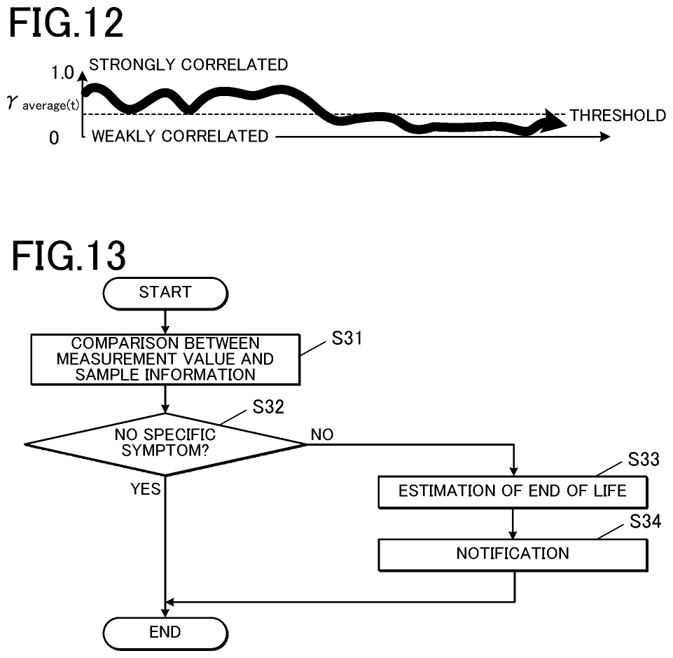

FIG. 12 is a conceptual diagram illustrating how the average value changes as it shifts from the non-terminal period to the terminal period. As illustrated in FIG. 12, the value becomes smaller as it approaches the terminal period due to decline in vital functions. In the above-mentioned step S23, if the average value becomes equal to or less than the predetermined threshold, it may be determined that the correlation has collapsed. Further, in the above-mentioned step S25, it may be determined that the physical condition has deteriorated due to a change in value.

According to the above-described correlation information comparison processing, since the data of the individual subject person is used, medical workers can accurately determine that the terminal period has approached due to decline in vital functions of the subject person.

Further, since the determination is feasible with only two vital values, processing can be performed more easily.

### <Specific Symptom Detection Processing>

The specific symptom detection processing is processing for estimating that the end of life is approaching from the specific symptom occurring in each vital value of the subject person.

The specific symptom detection processing may be executed as supplementary information for the correlation information comparison processing described above.

FIG. 13 is a flowchart illustrating an exemplary flow of the specific symptom detection processing.

As illustrated in FIG. 13, when the controller 21 receives vital values measured and transmitted by the measurement device 10, the controller 21 compares the vital values (measurement values) with sample information stored in the sample information storage device 224 (step S31) and determines the presence of the occurrence of a specific symptom in the subject person (step S32).

Further, if it is determined that no specific symptom has occurred (YES in step S32), the controller 21 terminates the present processing.

On the other hand, if it is determined that a specific symptom has occurred (NO in step S32), the controller 21 calculates a value for defining an occurrence status of the specific symptom and estimates the end of life of the subject person based on the calculated value (step S33).

FIG. 14 is a diagram illustrating an exemplary state in the terminal period.

Examples of the specific symptom include, for example, abnormal respiration rhythm (Cheyne-Stokes breathing, Biot's breathing, Kussmaul breathing, mandibular breathing, etc.), abnormal pulse (tachycardia, bradycardia, arrhythmia, etc.), abnormal body temperature (hypothermia, hyperthermia, etc.), abnormal blood pressure (hypertension, hypotension, etc.), pupil dilation, and consciousness disorder.

As illustrated in FIG. 14, these specific symptoms begin to occur when entering the terminal period, and these symptoms become more prominent as the death stage comes close.

For example, when an occurrence of abnormal respiration rhythm, abnormal pulse, abnormal body temperature, or abnormal blood pressure is determined, the controller 21 calculates an occurrence rate thereof per unit time (a value for defining the occurrence status of the specific symptom).

Further, when an occurrence of pupil dilation is determined, the controller 21 calculates a pupil enlargement ratio relative to the size of the pupil at the time of health (a value for defining the occurrence status of the specific symptom).

Further, when an occurrence of consciousness disorder is determined, the controller 21 calculates an occurrence rate thereof per unit time (a value for defining the occurrence status of the specific symptom).

Further, when the calculated value has exceeded s predetermined threshold, the controller 21 estimates that it indicates a predetermined number of days until deathbed. The number of days until deathbed can be changed according to the threshold to be set.

In addition, since the specific symptom occurring in the terminal period is variable in appearance pattern depending on each disease, the determination can be optimized in light of disease information prepared in advance. For example, as respective characteristic patterns, senility worsens little by little, heart disease repeats worsening and recover, and cancer rapidly worsens in the terminal period.

Further, based on the disease information, since there is a vital value relationship that is easy or difficult to change, the state of the disease can be checked by measuring the correlation between vital values.

Next, the controller 21 causes the display device 23 to display a message or notifies this by flickering light or issuing an alarm (step S34), and terminates the present processing.

According to the specific symptom detection processing, the end of life (severity) of each subject person can be predicted without preparing in advance comparison data (for example, a result of occurrence of a severe symptom of the subject person). Especially, in the terminal period, the entire duration may be a short period and accordingly generating comparison data for each individual subject person may be difficult. In this respect, the processing requiring no comparison data is useful.

Further, according to the specific symptom detection processing, it is possible to prevent such a situation that a subject person suddenly dies at timing unpredicted by surrounding people, for example, at a home or any site where no specialist is present, or at a nursing facility or a chronic-stage hospital or other comparable sites where there are specialists (doctors and nurses) but there are many patients per specialist and accordingly the change in vital values tends to be overlooked.

### [Effects of the Present Embodiment]

As described above, in the health condition determination system 100 according to the present embodiment, the controller 21 of the management device 20 generates the model information indicating daily circadian rhythm of the subject person from the vital value for one day or more measured by the measurement device 10 that measures the vital value of the subject person. Further, the controller 21 compares the vital value measured by the measurement device 10 after generation of the model information with the model information and determines the presence of a disturbance occurrence in the circadian rhythm of the subject person based on the comparison result.

Therefore, whether the health condition is normal or abnormal is determined using the data of the individual subject person, and accordingly the determination can be performed accurately.

Further, since the determination is feasible by using only one vital value, the processing is easy. Further, only one day is enough to generate model information for comparison, the processing is easy.

Accordingly, the health condition of a subject person can be determined more accurately and simply.

Further, according to the present embodiment, the controller 21 compares the vital value measured by the measurement device 10 at every predetermined timing with data of the model information at a time corresponding to the predetermined timing, and determines the presence of a disturbance occurrence in the circadian rhythm of the subject person based on the difference value.

Therefore, the determination whether the health condition is normal or abnormal can be performed at every predetermined timing.

Further, since a disturbance in circadian rhythm occurs due to a decline in vital functions, the circadian rhythm can be used as an index for determination of the terminal period.

Further, according to the present embodiment, the controller 21 compares a daily vital value measured by the measurement device 10 with the model information to calculate a deviation amount from the model information per day, and determines the presence of a disturbance occurrence in the circadian rhythm of the subject person based on the deviation amount.

Therefore, it can be determined whether the health condition is normal or abnormal every day.

Further, according to the present embodiment, the measurement device 10 measures at least two types of vital values, and the controller 21 generates the second model information indicating the correlation between the vital values from the at least two types of vital values. Further, the controller 21 generates the correlation information indicating the correlation between the at least two types of vital values measured by the measurement device 10 after generation of the second model information, and compares the generated correlation information with the second model information to calculate the comparison result.

Therefore, the correlation information indicating the correlation between at least two types of vital values can be used as the index value indicating the degree of decline in vital functions. Further, since the degree of decline in vital functions of the subject person can be grasped by using the data of each individual subject person, medical workers can perform determination accurately. Further, since the determination is feasible by using only two vital values, the processing is easy.

Accordingly, it is possible to grasp the degree of decline in vital functions of each subject person more accurately and simply.

Further, according to the present embodiment, the controller 21 calculates an average value of correlation values generated from each of two-types of vital values in a plurality of different combinations, and notifies that the average value is equal to or less than the threshold if the average value is equal to or less than the threshold.

Therefore, when the calculated average correlation value is equal to or less than the threshold, notifying medical workers of this can prevent oversight of determination timing or the end of life. Further, determination accuracy can be further enhanced by using an average value of a plurality of correlation values.

Further, according to the present embodiment, the controller 21 compares the vital value measured by the measurement device 10 with vital value sample information indicating a specific symptom at the terminal period acquired in advance, and determines the presence of the specific symptom occurring in the subject person.

Therefore, when a subject person is in the terminal period, the severity of the subject person can be predicted without preparing personal comparison data in advance.

Further, according to the present embodiment, the controller 21 calculates a value for defining the occurrence status of the specific symptom from the vital value measured by the measurement device 10, and estimates the number of days until the death of the subject person based on the calculated value.

Therefore, it is possible to prevent a situation that the subject person suddenly dies at timing unpredicted by surrounding people.

### [Miscellaneous]

Embodiments to which the present invention can be applied are not limited to the above-described embodiments, and can be appropriately changed without departing from the spirit of the present invention.

For example, the storage device 22 and the display device 23 may be provided separately from the management device 20. Further, the vital values measured by the measurement device 10 may be transmitted to the management device 20 via a terminal device, such as a smartphone or a tablet terminal.

Although embodiments of the present invention have been described and illustrated in detail, the disclosed embodiments are made for purposes of illustration and example only and not limitation. The scope of the present invention should be interpreted by terms of the appended claims

What is claimed is:

1. A health condition determination system comprising:
a measurement device that measures a vital value of a subject person; and
a hardware processor that generates model information indicating daily circadian rhythm of the subject person from the vital value measured by the measurement device for one day or more, compares a vital value measured by the measurement device after generation of the model information with the model information, and determines the presence of a disturbance occurrence in the circadian rhythm of the subject person based on the comparison result.

2. The health condition determination system according to claim 1, wherein the hardware processor compares a vital value measured by the measurement device at each predetermined timing with data of the model information at a time corresponding to the predetermined timing, and determines the presence of a disturbance occurrence in the circadian rhythm of the subject person based on the difference value.

3. The health condition determination system according to claim 1, wherein the hardware processor compares a daily vital value measured by the measurement device with the model information to calculate a deviation amount from the model information per day, and determines the presence of a disturbance occurrence in the circadian rhythm of the subject person based on the deviation amount.

4. The health condition determination system according to claim 1, wherein
the measurement device measures at least two types of vital values, and
the hardware processor generates second model information indicating a correlation between the at least two types of vital values, from the at least two types of vital values measured by the measurement device, generates correlation information indicating the correlation between the at least two types of vital values measured by the measurement device after generation of the second model information, and compares the generated correlation information with the second model information to calculate the comparison result.

5. The health condition determination system according to claim 4, wherein the hardware processor calculates an average value of correlation values generated from each of two-types of vital values in a plurality of different combinations, and notifies that the average value is equal to or less than a threshold if the average value is equal to or less than the threshold.

6. The health condition determination system according to claim 1, wherein the hardware processor compares the vital value measured by the measurement device with vital value sample information indicating a specific symptom at the terminal period acquired in advance, and determines the presence of the specific symptom occurring in the subject person.

7. The health condition determination system according to claim 6, wherein the hardware processor calculates a value for defining the occurrence status of the specific symptom from the vital value measured by the measurement device, and estimates the number of days until deathbed of the subject person based on the calculated value.

8. A health condition determination method comprising:
generating model information indicating daily circadian rhythm of a subject person from a vital value measured for one day or more by a measurement device that measures the vital value of the subject person; and
comparing a vital value measured by the measurement device after generation of the model information with the model information and determining the presence of a disturbance occurrence in the circadian rhythm of the subject person based on the comparison result.

9. A non-transitory recording medium that stores a computer-readable program that causes a computer to function as:
a hardware processor that generates model information indicating daily circadian rhythm of a subject person from a vital value measured for one day or more by a measurement device that measures the vital value of the subject person; compares a vital value measured by the measurement device after generation of the model information with the model information; and determines the presence of a disturbance occurrence in the circadian rhythm of the subject person based on the comparison result.
